(19) United States
(12) Patent Application Publication
(10) Pub. No.: US 2014/0127645 A1
(43) Pub. Date: May 8, 2014

(54) MEDICAL AND DENTAL IMPLANT PACKAGE

(71) Applicants: Gershon Goldenberg, Pardes Hana Karkur (IL); Eitan Shtarkman, Kadima (IL); Pinchas Kfir, Petach-Tikva (IL)

(72) Inventors: Gershon Goldenberg, Pardes Hana Karkur (IL); Eitan Shtarkman, Kadima (IL); Pinchas Kfir, Petach-Tikva (IL)

(73) Assignee: Precise Implant Systems E.S. Ltd., Or Akiva Industrial Park (IL)

(21) Appl. No.: 14/130,154

(22) PCT Filed: Jun. 28, 2012
(86) PCT No.: PCT/IL2012/000266

§ 371 Date: Dec. 30, 2013

Related U.S. Application Data
(60) Provisional application No. 61/502,882, filed on Jun. 30, 2011.

(51) Int. Cl. A61C 8/00; A61B 19/02; A61B 17/86

(52) U.S. Cl. CPC A61C 8/0087; A61C 8/0089; A61C 8/0022; A61B 17/86; A61B 19/026
USPC 433/174; 606/301; 206/438

(57) ABSTRACT

The invention is a protective package for delivering a medical or dental implant to an implantation site. The protective package protects the implant from contact with any surfaces from which bacteria or other contaminants could be transferred to the implant itself. The protective package is adapted to allow the implant to be delivered to the implantation site and to allow the primary retention of the implant in a bone or other type of tissue to be carried out before completely removing the implant from the protective packaging.

### FIELD OF THE INVENTION

The present invention relates to medical implants, and, more particularly, to packages for housing and delivery systems of medical and dental implants.

### BACKGROUND OF THE INVENTION

Generally, medical and dental implants are inserted into a body to restore the function of or replace the anatomy of a patient. One of the most common complications of transplant procedures is infection that results from introduction of foreign matter or bacteria into the site of the implant during the procedure. To limit the possibility that the implant itself will be the cause of infection, the implants, and components for use therewith, are supplied by the manufacturers in sterile, sealed packages. These packages are delivered to the operating room at which the implant is to be installed where they are opened and the implant removed by the medical staff. Once removed from their packages the implants are exposed to the environment and to contact with the gloved hands of the staff that handle it. Both the environment and gloves can be seriously contaminated by bacteria and organic matter that results from the preliminary surgical steps that are carried out to prepare the location in the body for the installation of the implant. The problem is especially acute in the case of dental implants, since as the oral surgeon moves the implant into the oral cavity it is very difficult to prevent contact with the tongue, lips, saliva, etc., all of which are known to be hosts to very large populations of bacteria.

It is therefore a purpose of the present invention to provide an implant package and delivery system that provides maximum sterile and clean accessibility of an implant to the implantation site.

It is another purpose of the present invention to provide an implant package and delivery system that allows the implant to be delivered to the implantation site within a protective package which is completely removed only after the implant is at least partially installed at the implantation site.

Further purposes and advantages of this invention will appear as the description proceeds.

### SUMMARY OF THE INVENTION

In a first aspect the invention is a protective package for delivering a medical or dental implant to an implantation site in a bone or other type of tissue. The protective package protects the implant from contact with any surfaces from which bacteria or other contaminants could be transferred to the implant itself during delivery to the implantation site and can also be used in achieving primary retention of the implant in the bone or other type of tissue. The protective package comprising a sealed outer covering and an implant protector housing shaped and dimensioned to provide an enclosure that entirely surrounds an implant and partially surrounds an implant carrier whose top portion projects outside of the top of the implant protector housing and whose bottom portion is reversibly engaged with the top of the implant.

The bottom of the implant protector housing is open. The top of the implant protector housing is adapted to provide an airtight seal around the top portion of the implant carrier that projects outside of the top of the implant protector housing and is adapted to allow the top portion of the implant carrier to be moved into and out of the implant protector housing. The implant protector housing is further adapted to engage the implant carrier when the implant carrier is pushed down into the implant protector housing far enough to cause a part of the implant to be pushed through the open bottom of the implant protector housing, whereupon rotation of the implant protector housing causes the implant carrier and the implant to be rotated together about their common longitudinal axis as a single unit, thereby allowing the primary retention of the implant in the bone or other type of tissue to be carried out before completely removing the implant from the protective packaging.

In embodiments of the protective package of the invention the top of the implant protector housing is adapted to allow the top portion of the implant carrier to be slid into and out of the implant protector housing by manually pressing on the top of the implant carrier.

In embodiments of the protective package of the invention the top of the implant protector housing comprises a cap, which when rotated causes a mechanism within the implant protector housing to advance the implant carrier into the interior of the implant protector housing and to push on the top of the implant, expelling the implant through the bottom of the implant protector housing.

Embodiments of the protective package of the invention comprise one or more vials or capsules connected in series with the implant protector housing by friction or by screwing the two parts together, the vials or capsules shaped and dimensioned to contain implant accessories.

In a second aspect the invention is a method for implanting a medical or dental implant that is supplied in a protective package according to the first aspect of the invention in a previously prepared implantation site in a bone or other type of tissue.

The method of the invention comprises:
a) removing the sealed outer covering from the protective package;
b) grasping the implant protector housing;
c) moving the implant protector housing and positioning it above the previously prepared implantation site in the bone or other type of tissue;
d) lowering the implant protector housing until its open end is as close as possible to the implantation site;
e) pushing down on the top of the implant carrier while firmly grasping the top of the implant protector housing until the implant is partially expelled through the bottom of the implant protector housing and contacts the bone or other type of tissue at the implantation site;
f) threading the implant into the bone or other tissues until primary retention of the implant in the bone or other tissues has been achieved;
g) pulling up on the implant protector housing and the implant carrier and removing them from the implantation site; and
h) screwing the implant into its final depth in the bone or other tissues with the aid of one of a ratchet wrench, a torque wrench, or a surgical motor.

In embodiments of the method of the invention step f) is carried out by a surgeon pushing down on the top of the implant carrier and at the same time rotating the implant protector housing with his fingers, thereby manually, threading the implant into the bone or other type of tissue and achieving primary retention of the implant.

In embodiments of the method of the invention step is replaced with the steps of:
f-1) pushing down on the top of the implant carrier and rotating the implant carrier with the aid of one of a ratchet wrench, a torque wrench, or a surgical motor, thereby, threading the implant into the bone or other type of tissue and achieving primary retention of the implant; and
f-2) separating the ratchet wrench, torque wrench, or surgical motor from the top of the implant carrier.

All the above and other characteristics and advantages of the invention will be further understood through the following illustrative and non-limitative description of embodiments thereof, with reference to the appended drawings. In the drawings the same numerals are used to indicate like elements in different drawings.

### BRIEF DESCRIPTION OF THE DRAWINGS

FIG. 1 is an exploded view showing the components of an embodiment of a dental implant package according to the invention;

FIG. 2A is a view showing the dental implant package of FIG. 1 as it would be delivered to the surgeon performing the implant surgery;

FIG. 2B is a cross-sectional view in plane A-A of FIG. 2A;

FIG. 3 shows the separation of the implant protector housing from the accessory vial;

FIG. 4 shows the implant inside the implant protector as it is brought to the implantation site in a jaw bone of the patient;

FIG. 5 symbolically shows the implant partially expelled from the implant protector in order to begin attaching it to the bone;

FIG. 6A symbolically shows the how the primary retention of the implant in the bone;

FIG. 6B is a cross-sectional view in plane A-A of FIG. 6A;

FIG. 7A symbolically shows the separation of the implant carrier from the implant after the primary retention of the implant in the bone;

FIG. 7B is a cross-sectional view in plane A-A of FIG. 7A; and

FIG. 8 symbolically shows a wrench being attached to the implant in order to complete the procedure.

### DETAILED DESCRIPTION OF EMBODIMENTS OF THE INVENTION

The invention is a protective package for delivering a medical or dental implant to an implantation site while protecting the implant from contact with any surfaces from which bacteria or other contaminants could be transferred to the implant itself. The invention accomplishes its purpose by providing packaging that allows the implant to be delivered to the implantation site and for the primary retention of the implant in a bone or other type of tissue to be carried out before removing the implant from the protective packaging. The invention will be descried herein with reference to a specific embodiment for packaging a dental implant; however the invention is not limited to this particular embodiment and can be easily adapted mutandis mutatis for any other type of medical or dental implant.

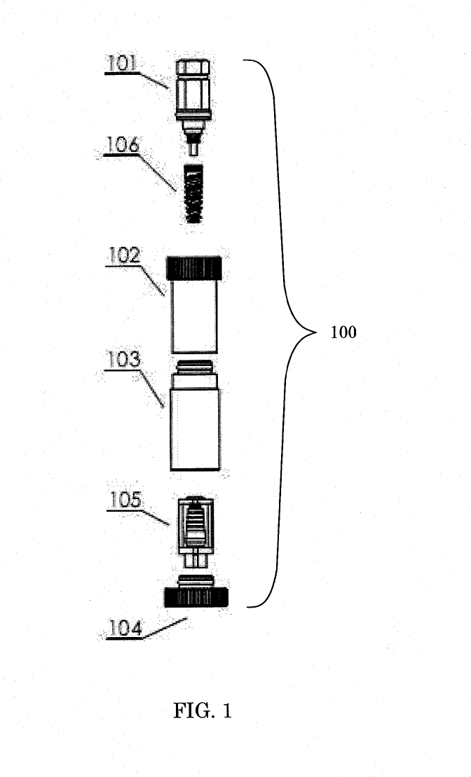

FIG. 1 is an exploded view showing the main components of an embodiment of a dental implant package 100 according to the invention. Package 100 is comprised of the following components: implant carrier 101, implant 106, implant protector housing 102, accessory vial 103, vial cover 104, and accessory mount 105. Accessory vial 103, vial cover 104, and accessory mount 105 are included in the figures to illustrate how other implant accessories such as an implant abutment, healing screw, or healing cover can be supplied together with the implant in a single sterile package in one or more vials or capsules that are connected in series with the implant protector housing by friction or by screwing the two parts together.

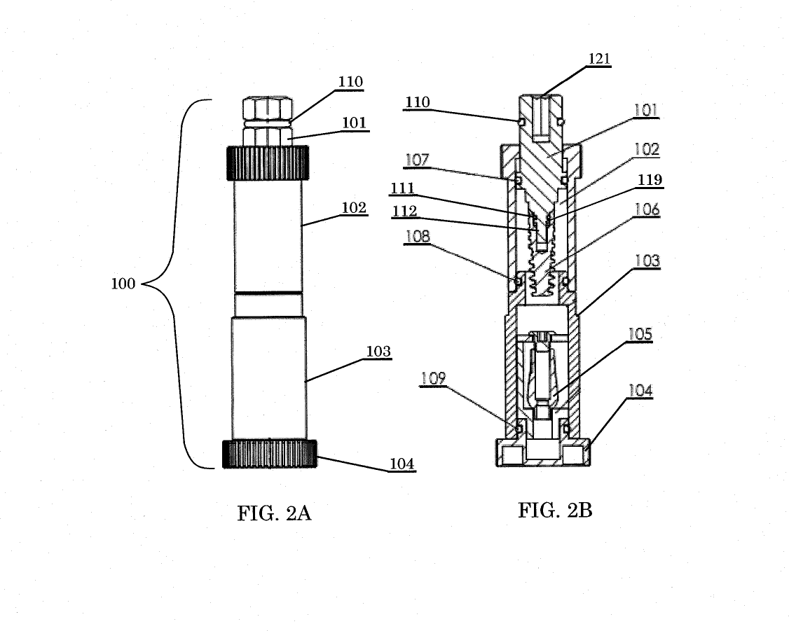

At the manufacturing facility all components of dental implant package 100 are sterilized and assembled as shown in FIG. 2A. The assembled implant package is then inserted into a sealed outer covering to further protect its sterility during shipping and storage prior to use. In the operating room or clinic the outer covering is removed and dental implant package 100 is handed to the surgeon who performs the implant procedure as described herein below.

FIG. 2B is a cross-sectional view in plane A-A of FIG. 2A. In addition to the components previously identified in FIG. 1, socket 121 in the head of implant carrier 101, O-rings 107-111, and neck 112 at the end of the implant carrier 101 are shown. Socket 121, which has for example a hexagonal shape, allows implant carrier 101 to be elongated by use of an elongating device that connects to a wrench or to an implant screwdriver. This allows implant carrier 101 and implant 106 to be rotated together about their common longitudinal axis as a single unit as will be described herein below. The function of neck 112 will be described herein below. O-ring 109 has the dual function of creating an airtight seal between vial cover 104 and the interior of accessory vial 103 holding the two parts together while maintaining the sterility of the interior of the accessory vial and also allowing the cover to be pulled off the vial when desired. O-ring 108 serves a similar function between implant protector housing 102 and accessory vial 103. O-ring 107 and O-ring 110 maintain a seal between implant carrier 101 and the interior wall of implant protector housing 102, while allowing the implant carrier to slip up and down within the carrier.

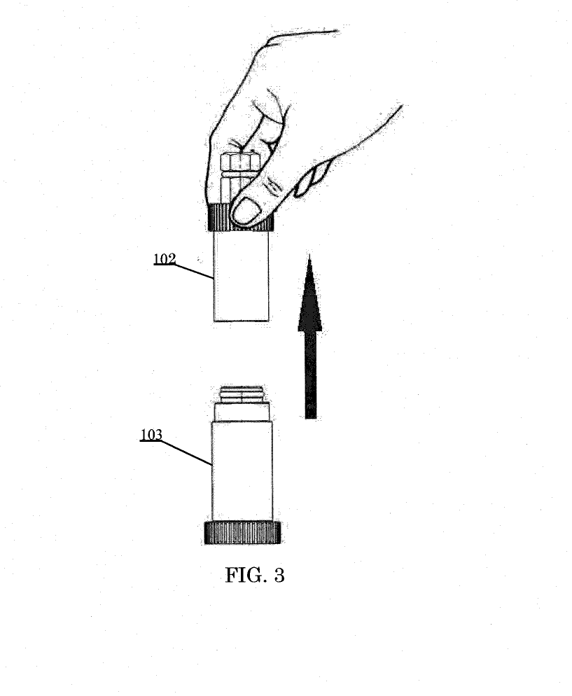

The implantation procedure will now be described with reference to the figures. When dental implant package 100 is delivered to the oral surgeon the sealed outer covering is removed and then the implant protector housing 102 is separated from the accessory vial 103 by pulling them in opposite directions along the longitudinal axis as shown in FIG. 3. Accessory vial 103 with the enclosed accessory mount 105 is then put aside for later use.

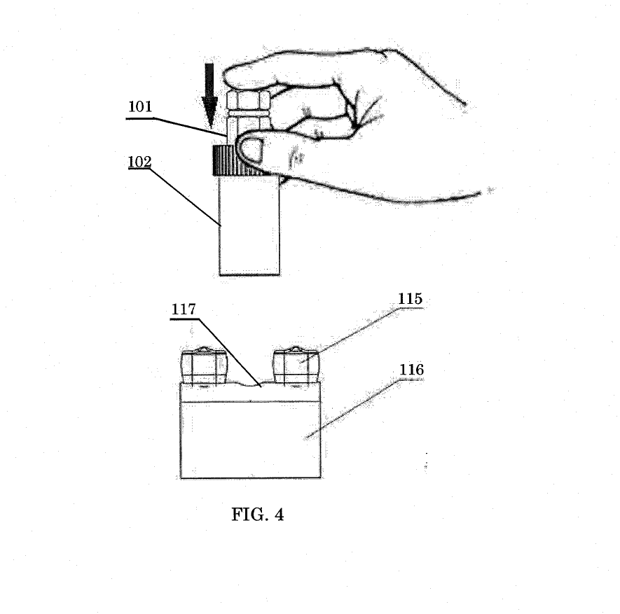

The oral surgeon grasps implant protector housing 102 in his hand and moves it inside the mouth of the patient positioning it above the previously prepared implantation site 117, which in this example is between two teeth 115 in a portion of a jaw 116 of the patient (see FIG. 4). The surgeon lowers the implant protector housing 102 until its open end is as close as possible to the implant site. Then, he pushes down on the top of implant carrier 101 with his index finger while firmly grasping the top of implant protector housing 102 (the outer surface may be knurled as shown or roughened in another manner to improve the grip) between his thumb and other fingers as shown in FIG. 4.

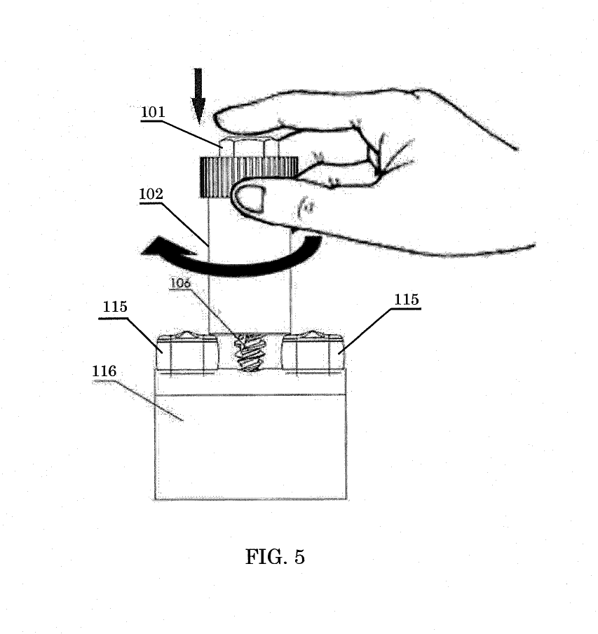

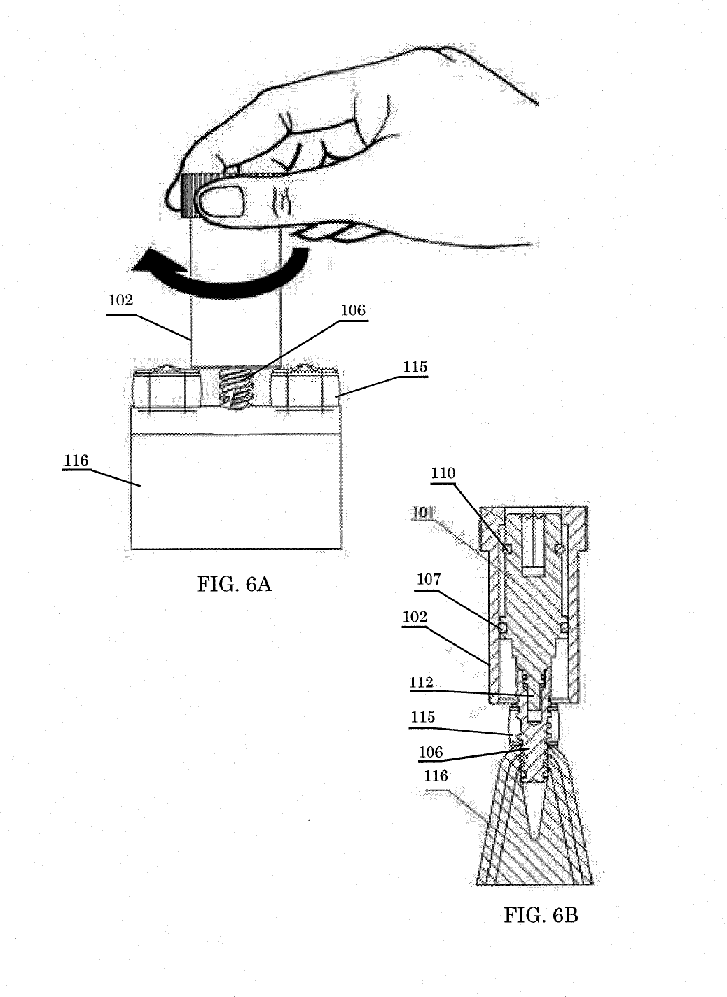

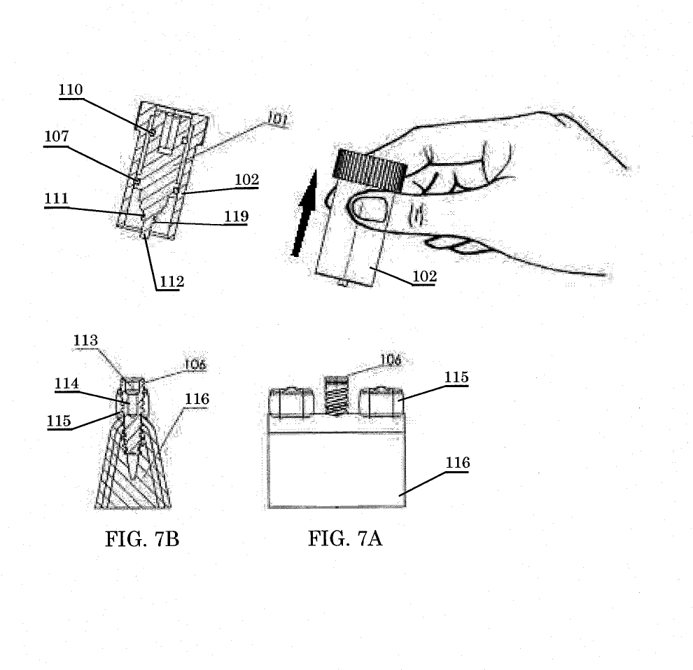

As shown in FIG. 5, as the implant carrier 101 is pushed into the top of the implant protector housing 102, the implant is expelled through the bottom. It is only at this stage that the implant is exposed to the surrounding tissue and fluids and at no stage has its surface been touched by either the surgeon or any other member of the operating team. The head of implant carrier 101 has a shape, e.g. hexagonal, that matches that of a socket in the interior of the implant protector housing 102 so that if the implant protector housing 102 is turned as shown by the broad curved arrow in FIG. 5, then implant carrier 101 will turn with it. When the implant carrier 101 is pushed against implant 106 inside implant protector housing 102 as shown in FIG. 2B (and more easily understood by referring to FIG. 6B and FIG. 7B) a neck 112 at the lower end of implant carrier 101, slidingly fits into bore 114 on the upper end of implant 106 and a shoulder 119 at the top of neck 112 enters a socket 113 (FIG. 7B) at the top end of implant 106. O-ring 111 insures a tight fit while allowing shoulder 119 of neck 112 to be easily pushed into and pulled out of socket 113 in the head of implant 106. The configuration of shoulder 119 and socket 113, which have matching shapes, e.g. hexagonal, allows the implant carrier 101 and implant 106 to be rotated together about their common longitudinal axis as a single unit.

Thus by pushing down on the top of implant carrier 101 and at the same time rotating implant protector housing 102 as shown in FIGS. 5 and 6A, the surgeon begins to manually thread the dental implant 106 into the jaw 116 of the patient. FIG. 6B is a cross-sectional in a longitudinal plane bisecting FIG. 6A. In this figure it can be seen how the lower threads on the dental implant 106 has been screwed into jaw 116 and how the implant carrier 101 and implant 106 fit together inside implant protector housing 102.

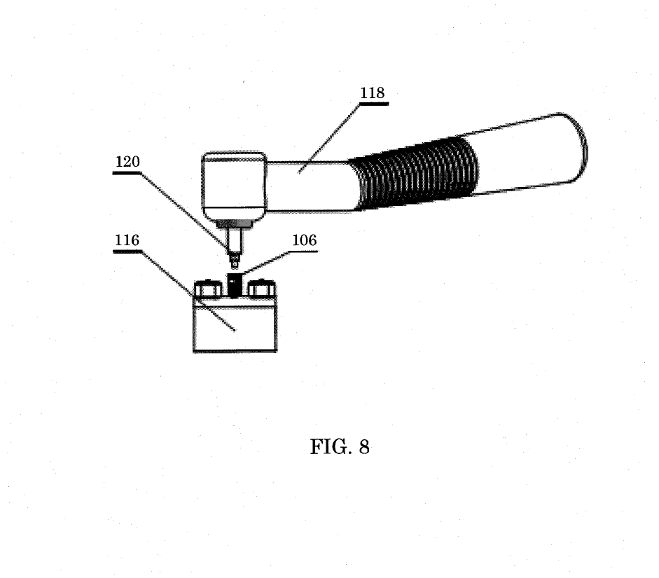

After the implant 106 has its primary retention in the bone, the surgeon can remove the implant protector housing 102 and the implant carrier 101 by pulling them up as shown in FIG. 7A and the cross-sectional view FIG. 7B. Finally, as shown in FIG. 8, the surgeon screws the implant into its final depth in the bone by inserting the bit or adapter 120 of a ratchet wrench, torque wrench, or surgical motor into socket 113 at the top end of implant 106 as known in the art.

Alternatively, the primary retention in a bone or other tissue can be achieved with the aid of a tool, comprised of a ratchet wrench, torque wrench, or surgical motor, after which said tool is withdrawn to allow the implant protector housing 102 and the implant carrier 101 to be removed and then the bit or adapter of said tool is reinserted into socket 113 at the top end of implant 106 and the surgeon screws the implant into its final depth in the bone.

In an alternative embodiment not shown in the figures, the top of the implant protector housing 102 comprises a cap, which when rotated causes a mechanism within said implant protector housing to advance implant carrier 101 into the interior of implant protector housing 102 and to push on the top of implant 106, expelling the implant through the bottom of the housing.

Although embodiments of the invention have been described by way of illustration, it will be understood that the invention may be carried out with many variations, modifications, and adaptations, without exceeding the scope of the claims.

1. A protective package for delivering a medical or dental implant to an implantation site in a bone or other type of tissue while protecting the implant from contact with any surfaces from which bacteria or other contaminants could be transferred to the implant itself and for use in achieving primary retention of said implant in said bone or other type of tissue; said protective package comprising a sealed outer covering and an implant protector housing shaped and dimensioned to provide an enclosure that entirely surrounds an implant and partially surrounds an implant carrier whose top portion projects outside of the top of said implant protector housing and whose bottom portion is reversibly engaged with the top of said implant;
wherein:
a) the bottom of said implant protector housing is open;
b) the top of said implant protector housing is adapted to provide an airtight seal around the top portion of said implant carrier that projects outside of the top of said implant protector housing, said top of said implant protector housing adapted to allow said top portion of said implant carrier to be moved into and out of said implant protector housing; and
c) said implant protector housing is further adapted to engage said implant carrier when said implant carrier is pushed down into said implant protector housing far enough to cause a part of said implant to be pushed through said open bottom of said implant protector housing, whereupon rotation of said implant protector housing causes said implant carrier and said implant to be rotated together about their common longitudinal axis as a single unit, thereby allowing the primary retention of said implant in said bone or other type of tissue to be carried out before completely removing said implant from said protective packaging.

2. The protective package of claim 1 wherein the top of the implant protector housing is adapted to allow the top portion of said implant carrier to be slid into and out of said implant protector housing by manually pressing on the top of said implant carrier.

3. The protective package of claim 1 wherein the top of the implant protector housing comprises a cap, which when rotated causes a mechanism within said implant protector housing to advance the implant carrier into the interior of said implant protector housing and to push on the top of the implant, expelling said implant through the bottom of said implant protector housing.

4. The protective package of claim 1 comprising one or more vials or capsules connected in series with the implant protector housing by friction or by screwing the two parts together, said vials or capsules shaped and dimensioned to contain implant accessories.

5. A method for implanting a medical or dental implant that is supplied in a protective package according to claim 1 in a previously prepared implantation site in a bone or other type of tissue;
said method comprising:
a) removing the sealed outer covering from said protective package;
b) grasping the implant protector housing;
c) moving said implant protector housing and positioning it above said previously prepared implantation site in said bone or other type of tissue;
d) lowering said implant protector housing until its open end is as close as possible to said implantation site;
e) pushing down on the top of the implant carrier while firmly grasping the top of said implant protector housing until the implant is partially expelled through the bottom of said implant protector housing and contacts said bone or other type of tissue at said implantation site;
f) threading said implant into said bone or other tissues until primary retention of said implant in said bone or other tissues has been achieved;
g) pulling up on said implant protector housing and said implant carrier and removing them from said implantation site; and
h) screwing said implant into its final depth in said bone or other tissues with the aid of one of a ratchet wrench, a torque wrench, or a surgical motor.

6. The method of claim 5 wherein step f) is carried out by a surgeon pushing down on the top of the implant carrier and at the same time rotating the implant protector housing with his fingers, thereby manually, threading the implant into the bone or other type of tissue and achieving primary retention of said implant.

7. The method of claim 5 wherein step f) is replaced with the steps of:
f-1) pushing down on the top of the implant carrier and rotating said implant carrier with the aid of one of a ratchet wrench, a torque wrench, or a surgical motor, thereby, threading the implant into the bone or other type of tissue and achieving primary retention of said implant; and
f-2) separating said ratchet wrench, torque wrench, or surgical motor from the top of said implant carrier.
